(19) United States
(12) Patent Application Publication
(10) Pub. No.: US 2014/0364325 A1
(43) Pub. Date: Dec. 11, 2014

(54) Array of Sensors Functionalized with Systematically Varying Receptor Materials

(71) Applicant: MATRIX SENSORS, INC., Pleasanton, CA (US)

(72) Inventors: Michael D Cable, Danville, CA (US); Heinrich Sussner, Palo Alto, CA (US)

(21) Appl. No.: 13/911,003

(22) Filed: Jun. 5, 2013

(51) Int. Cl. G01N 33/50

(52) U.S. Cl. CPC G01N 33/50
USPC 506/7; 506/15; 506/18; 506/17; 506/14

(57) ABSTRACT

A sensor array includes resonator sensors having respective receptor materials disposed thereon. The receptor materials have a physical property relevant to their ability to bind or adsorb one or more analytes in a sample. The physical property of the receptor materials on the sensors systematically increases or decreases in degree from one sensor to the next in the array. The device also comprises at least one detector for detecting sensor responses when masses of the analytes are adsorbed or bound to the receptor materials on the sensors. With this graded panel of sensors in the array, the analytes may adsorb or bind to the functionalized sensors with a pattern of responses specific to each analyte.

### BACKGROUND

The invention relates to sensor arrays for detecting analytes, and in particular to an array of sensors functionalized with systematically varying receptor materials for detecting one or more analytes.

Resonant sensors use target molecules adsorbed in the sensing material to change properties that are reflected in the resonance frequency. A wide variety of cantilever, membrane and piezoelectric resonator-based sensors have been fabricated using MEMS technology. These sensors generally detect agents through the use of polymer films and coatings with selective adsorption for a specific agent or set of agents. Although these sensors provide a certain degree of sensitivity, it is desirable in many applications to have sensors with even higher sensitivities.

A capacitive micromachined ultrasonic transducer (cMUT) is a micromachined device having a substrate and a membrane supported above the substrate by an insulating material. A variable voltage applied between the substrate and membrane drives the membrane to vibrate and emit sound waves at ultrasonic frequencies. Arrays of cMUTs have been used for transmitting and receiving ultrasonic beam patterns in air and water over a frequency range from 10 kHz to 100 MHz. These cMUTs rely on the very large electric field (E>10⁸ V/m) in the gap of the capacitor to provide an electromechanical coupling coefficient close to unity.

cMUTs are mostly used for medical imaging. In addition, they have been used to indirectly measure various fluid characteristics, based on processing of ultrasonic signals transmitted and received through the fluid. In current cMUT devices and applications, the cMUT elements are used to transmit and/or receive ultrasonic energy between the cMUT element and the environment. Moreover, to ensure reliable and consistent operation, cMUT element membranes are normally designed to be non-reactive to chemicals, light, and other environmental factors that may alter or interfere with their operational characteristics. However, due to their resonant character, cMUT devices have the potential to be used as sensors, in a manner similar to MEMS cantilever, membrane, and piezoelectric resonator-based sensors.

U.S. Pat. No. 7,305,883 to Khuri-Yakub discloses arrays of sensors. Sensor elements include a functionalized membrane supported over a substrate by a support frame. The sensor element is connected to an electrical circuit, which is configured to operate the sensor element at or near an open circuit resonance condition. The mechanical resonance frequency of the functionalized membrane is responsive to binding of an agent to the membrane. The exterior surface of each sensor membrane is chemically functionalized to have an affinity for one or more specific, predetermined chemicals. A detector provides a sensor output responsive to the mechanical resonance frequency of the sensor element.

US patent application 2010/0180673 to Cable and Steiert discloses a method for analyzing liquid samples by applying a liquid to a cMUT device having an array of sensors, drying the sensors, and electronically detecting an agent bound to each of the plurality of sensors. An electrical circuit provides a sensor output responsive to a mechanical resonance frequency of the sensor. The exterior surface of sensor membrane is chemically functionalized to have an affinity for one or more specific, predetermined chemicals. The mechanical resonance frequency of the sensor is responsive to the binding of an agent to the functionalized membrane, and the mass of the agent bound to each of the sensors may be determined.

### SUMMARY

According to one aspect, a device for detecting one or more analytes comprises a sensor array including at least three resonator sensors having respective receptor materials disposed thereon. The receptor materials have a physical property relevant to their ability to bind or adsorb one or more of the analytes. The physical property of the receptor materials systematically increases or decreases in degree from the receptor material on a first one of the sensors, to the receptor material on a second one of the sensors, and to the receptor material on a third one of the at least three sensors. The device also comprises at least one detector for detecting sensor responses (e.g., changes in resonance frequencies) when masses of one or more of the analytes are adsorbed or bound to the sensors.

According to another aspect, a device for detecting one or more analytes comprises a sensor array including a plurality of resonator sensors having respective receptor materials disposed thereon. The sensors are arranged in rows and columns. The receptor materials have a first physical property relevant to their ability to bind or adsorb one or more of the analytes. The first physical property of the receptor materials on the sensors in at least one of the rows systematically increases or decreases in degree from the receptor material on a first one of the sensors in the row to the receptor material on a last one of the sensors in the row. Additionally, the first physical property or a second physical property of the receptor materials on the sensors in at least one of the columns systematically increases or decreases in degree from the receptor material on a first one of the sensors in the column to the receptor material on a last one of the sensors in the column. The device also comprises at least one detector for detecting sensor responses (e.g., changes in resonance frequencies) when masses of one or more of the analytes are adsorbed or bound to the sensors.

According to another aspect, a method comprises exposing a sensor array to a sample that potentially contains one or more analytes. The sensor array includes at least three resonator sensors having respective receptor materials disposed thereon. The receptor materials have a physical property relevant to their ability to bind or adsorb one or more of the analytes. The physical property of the receptor materials systematically increases or decreases in degree from the receptor material on a first one of the sensors, to the receptor material on a second one of the sensors, and to the receptor material on a third one of the at least three sensors. The method also comprises the steps of detecting sensor responses to the sample and determining from the sensor responses if the sample contains one or more of the analytes.

According to another aspect, a method comprises exposing a sensor array to a sample that potentially contains one or more analytes. The sensor array includes a plurality of resonator sensors having respective receptor materials disposed thereon. The sensors are arranged in rows and columns. The receptor materials have a first physical property relevant to their ability to bind or adsorb one or more of the analytes. The first physical property of the receptor materials on the sensors in at least one of the rows systematically increases or decreases in degree from the receptor material on a first one of the sensors in the row to the receptor material on a last one of the sensors in the row. Additionally, the first physical property or a second physical property of the receptor materials on the sensors in at least one of the columns systematically increases or decreases in degree from the receptor material on a first one of the sensors in the column to the receptor material on a last one of the sensors in the column. The method also comprises the steps of detecting sensor responses to the sample and determining from the sensor responses if the sample contains one or more of the analytes.

### BRIEF DESCRIPTION OF THE DRAWINGS

The foregoing aspects and advantages of the present invention will become better understood upon reading the following detailed description and upon reference to the drawings where:

FIG. 1 shows a schematic, cross-sectional view of a sensor according to one embodiment of the invention.

FIG. 2 shows a schematic cross-sectional view, of a sensor according to another embodiment of the invention.

FIG. 3 shows an example of an electrical circuit for a sensor according to some embodiments of the invention.

FIG. 4 shows a schematic, cross-sectional view of an array of sensors according to some embodiments of the invention.

FIG. 5 shows a schematic, plan view of an array of sensors according to some embodiments of the invention.

FIG. 6 is a schematic, plan view of an array of sensors having receptor materials that systematically increase in polarity according to some embodiments of the invention.

FIG. 7 is a graph showing masses adsorbed on sensors in an array according to some embodiments of the invention.

FIG. 8 is another graph showing masses adsorbed on sensors in an array according to some embodiments of the invention.

FIG. 9 is a flow chart showing steps of a method to determine the presence or amounts of one or more analytes according to some embodiments of the invention.

FIG. 10 shows a schematic, plan view of an array of sensors according to some embodiments of the invention.

FIGS. 11A-11D are graphs showing masses adsorbed on sensors in an array according to some embodiments of the invention.

FIG. 12 is a schematic, plan view of a sensor array having multiple rows and columns according to some embodiments of the invention.

FIG. 13 is a schematic, plan view of an array of sensors having receptor materials with systematic variation of increasing and decreasing polarity according to some embodiments of the invention.

### DETAILED DESCRIPTION OF PREFERRED EMBODIMENTS

In the following description, it is understood that all recited connections between structures can be direct operative connections or indirect operative connections through intermediary structures. A set of elements includes one or more elements. Any recitation of an element is understood to refer to at least one element. A plurality of elements includes at least two elements. Unless otherwise required, any described method steps need not be necessarily performed in a particular illustrated order. A first element (e.g. data) derived from a second element encompasses a first element equal to the second element, as well as a first element generated by processing the second element and optionally other data. Making a determination or decision according to a parameter encompasses making the determination or decision according to the parameter and optionally according to other data. Unless otherwise specified, an indicator of some quantity/data may be the quantity/data itself, or an indicator different from the quantity/data itself. Computer programs described in some embodiments of the present invention may be stand-alone software entities or sub-entities (e.g., subroutines, code objects) of other computer programs. Computer readable media encompass non-transitory media such as magnetic, optic, and semiconductor storage media (e.g. hard drives, optical disks, flash memory, DRAM), as well as communications links such as conductive cables and fiber optic links According to some embodiments, the present invention provides, inter alia, computer systems comprising hardware (e.g. one or more processors and associated memory) programmed to perform the methods described herein, as well as computer-readable media encoding instructions to perform the methods described herein.

The following description illustrates embodiments of the invention by way of example and not necessarily by way of limitation.

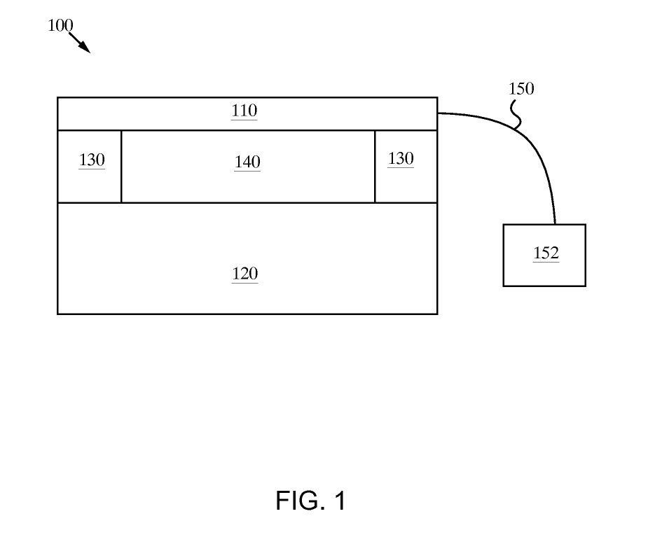

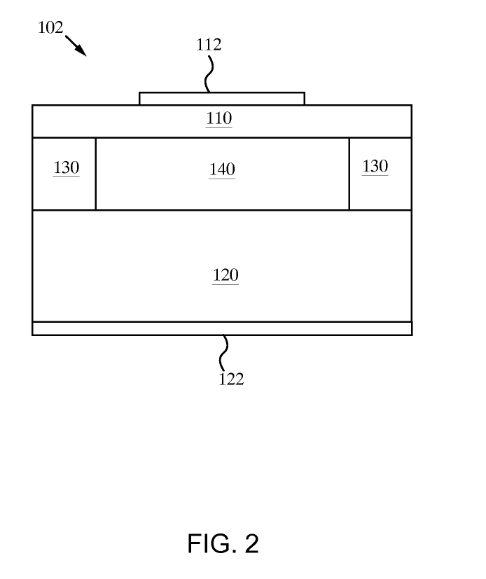

FIGS. 1 and 2 shows schematic cross-sectional diagrams of two examples of resonator sensors according to some embodiments of the invention. Resonator sensors include, without limitation, capacitive micromachined ultrasonic transducer (cMUT), cantilever, and piezoelectric resonator-based sensors. FIG. 1 shows a cMUT sensor 100 that has a functionalized membrane 110, which is functionalized with a receptor material for adsorbing or binding one or more analytes. Functionalized membrane 110 is supported over a substrate 120 by support frame 130. Functionalized membrane 110, support frame 130 and substrate 120 define a vacuum gap 140. Vacuum gap 140 is preferably between about 0.1 μm and about 5 μm in height. The sensor 100 is connected to a detector 150 through connector 152. In general, detector 152 preferably employs a detection modality to measure a sensor response (e.g., a change in the position or resonance frequency of the membrane 110) due to the mass of one or more analytes adsorbed or bound to the sensor 100. In preferred embodiments, detector 150 detects a resonance frequency of the functionalized membrane 110, which frequency may change due to the mass of one or more analytes adsorbed or bound to the receptor material on the sensor 100. Suitable detectors include, but are not limited to, an optical detector, a mechanical stress detector, a magnetic detector, and a capacitance detector.

In one embodiment, functionalized membrane 110 is driven thermally (by applied heat or by thermal noise) or electrically, and an optical detector is used to detect deflection or resonant frequency shifts of functionalized membrane 110. Interferometric optical detection techniques re described in U.S. Pat. No. 6,567,572, by Degertekin et al., which is incorporated herein by reference. In other embodiments, functionalized membrane 110 has thin piezoelectric or magnetic films that provide coupling. The resonant functionalized membranes 110 may be addressed by capacitor action (cMUTs), by a piezoelectric thin film (pMUTs), or by a magnetic film on the surface (mMUTs). Alternatively, a change in membrane deformation may be detected directly through a change in capacitance, or magnetic field, or piezoelectric signal, or change in resistance through the piezoresistive effect, or optically using an interferometer, or any other detection modality to measure the response of sensor 100 due to the mass of one or more analytes adsorbed or bound to the receptor material on the sensor 100, or due to a stiffening effect of the receptor material. Preferably, functionalized membrane 110 operates at a mechanical resonance frequency of at least about 1 MHz, more preferably between about 1 MHz and about 100 MHz.

FIG. 2 shows a cMUT sensor 102 having a functionalized membrane 110 that includes a first electrode 112. The substrate 120 contains a second electrode 122. Functionalized membrane 110 and substrate 120 are preferably thin membranes that are essentially parallel plate capacitors with a gap between the plates. In a preferred aspect of this embodiment, the conductive silicon wafer on which the functionalized membrane is fabricated, i.e. substrate 120, makes up one plate of the capacitor. A metal electrode 112 on top of the functionalized membrane 110 is the other plate of the capacitor. Functionalized membrane 110, which is supported by insulating support frame 130, is typically made of an insulating material, most commonly silicon, and is coated with the electrode 112. A low temperature oxide passivation layer may cover electrode 112 and functionalized membrane 110.

In some embodiments, functionalized membrane 110 is constructed to have a large surface area, for instance by adding vertical trenches, by making a portion of the membrane porous, or by adding cavities. Each of the cavities may be formed with a specific dimension based upon the desired resonant frequency. Each of the plurality of cavities is then configured to communicate with a common electrode, which thereby forms a single sensor. This way, it is possible to attach many more molecules of a species to the membrane and increase the mass loading or induced stress, and hence improve sensitivity.

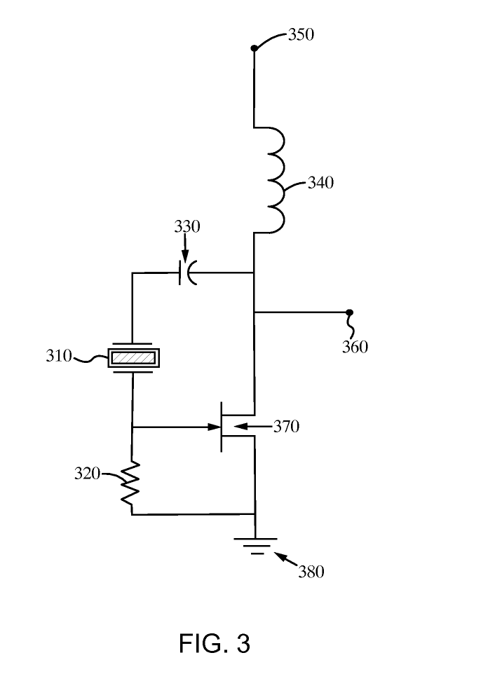

FIG. 3 shows one embodiment of a circuit suitable for use with a sensor. The circuit is one variation of a so-called Pierce oscillator. The circuit includes sensor element 310, resistor 320, capacitor 330, inductor 340, DC voltage source 350, sensor output 360, transistor 370, and connection to ground 380. Many other circuits are available to establish a resonant circuit using the sensor's resonant electrical input impedance. The output of these circuits is a sinusoidal signal whose frequency is the measurable quantity of interest.

In some embodiments, the sensor is placed in the feedback loop of an amplifier and the gain of the amplifier is adjusted such that the circuit oscillates. The frequency of the oscillator is tuned by adjusting the DC bias that is applied to the sensor element. By controlling this DC bias the resonance or oscillation frequency is placed near the open circuit resonant frequency of the sensor. This is done in order to reduce the noise in the oscillator circuit, and hence increase the sensitivity of the sensor. When analyte adsorbs or binds to the receptor material on the resonating element of the sensor (e.g., the membrane 110 or a cantilever), its open circuit resonance frequency shifts, and this imparts a frequency shift in the oscillator circuit. By measuring the resonance frequency of the oscillator, one can tell how much mass has deposited on the membrane 110.

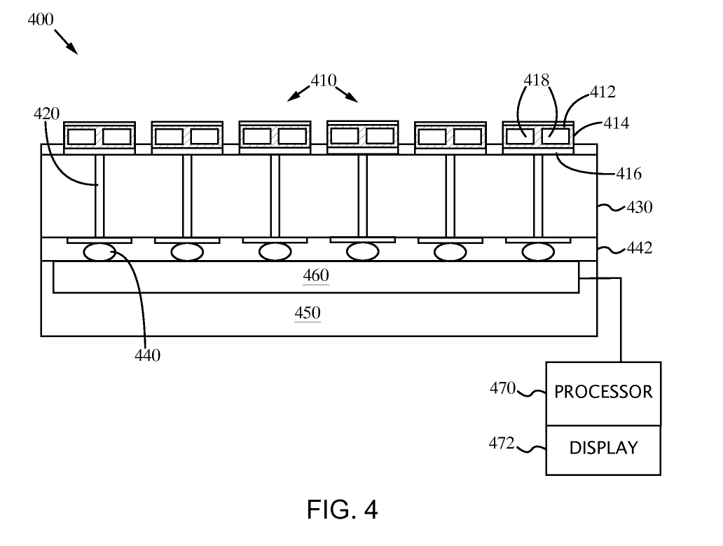

FIG. 4 is a schematic cross-sectional view of a sensor device 400 containing an array of sensors 410 according to an embodiment of the invention. In this example, each sensor 410 contains two sensor elements, each of which has a membrane 412, support frame 414, substrate 416 and vacuum gap 418. The exterior surface of each membrane 412 is chemically functionalized. The sensors are designed for sensitivity to mass loading and stress loading by analytes adsorbed or bound to the membrane 412, and for matching into the electronic circuitry such as a Pierce oscillator or any other type of oscillator that is used to detect the shift in the property of the membrane 412. Due to the functionalization of the membrane surface, analytes adsorb or bind to the surface of the membrane 412 when they are present in the environment or a sample to which the sensor is exposed. Consequently, the operational characteristics (e.g., impedance or resonance frequency) of the sensor will be altered, and this sensor response is detected.

The presence or amount of analyte(s) in a sample are measured by detecting the alteration of the operating characteristics of the resonating element (e.g., a membrane or cantilever). For example, an alteration in sensor characteristics can be detected by measuring the impedance of the sensor, or by measuring the change in the resonant frequency of the functionalized membrane 412. Interconnects 420 through wafer 430 provide electrical contacts from the sensors 410 to a wafer 450 with electronics layer 460. The interconnects 420 are separated from the electronics 460 by an underfill 442 and solder balls 440. (While solder bumps are shown in this figure, contacts may be made between wafer 430 and wafer 450 by any means known in the art, e.g. with an anisotropic conducting film).

The electronics layer 460 contains appropriate circuitry to drive and detect operational characteristics of the sensors 410, such as resonance frequencies of each membrane 412. Additional signal processing electronics or a processor 470 may be attached to the sensor electronics to further process the signals and to provide an indication of the presence or amount of analyte(s). For example, the indication of the presence or amount of analytes may be shown via the display 472 in communication (wirelessly or with wires) with the processor 470. The processor 470 receives data representative of the resonance frequencies (e.g., frequency output signals from the sensors 410) to determine the presence or amount of analyte(s). The processor 470 may be a microprocessor included with the device 400. Alternatively, processing functions may be performed in a separate processor or external computer in communication with the electronics layer 460. The external processor or computer receives data representative of the measured resonance frequencies and determines the presence or amount of analyte(s). Alternatively, multiple processors may be provided, e.g., providing one or more processors in the device 400 that communicate (wirelessly or with wires) with one or more external processors or computers.

Some processing of data can be done near the sensor. For instance, time averaging or multiplexing or digitization can be all processed in the vicinity of the sensor before being transmitted to a computer or a circuit board with a multiprocessor. Specific algorithms can be loaded in memory to perform the same functions one would in a digital computer and then drive displays where colored outputs can be used to indicate level of detection or hazard. As in many sensors deployed today, such as RF tags and implanted medical devices, it is possible to use RF antennas to couple and provide power to the sensor. Once a sensor is powered, it senses its function, and then the output of the sensor is re-radiated to a receiving antenna. In this fashion, the sensor device 400 can be passive and remotely addressed.

In some embodiments, a CMOS provides the circuitry to detect the mass loading of the membrane 412 either through an impedance change, by direct measurement, resonance frequency measurement, or any of various other means. The outputs of various sensors can be multiplexed, then a frequency counter can measure the frequencies. These outputs can then be digitized and stored and processed in a processor. The processor then can display the variation of the resonant frequency versus time and provide results of analysis of sensed species based, for example, on previously loaded models of sensitivity of multiple sensors to various chemicals.

The material properties and dimensions of the functionalized membranes 412 contribute to their resonant frequencies. In some embodiments, a DC bias is applied to the functionalized membranes 412 to maintain a very high electric field in the vacuum gaps 418. For instance, a silicon membrane 12 μm in diameter and 0.4 μm thick may resonate at a frequency of 42 MHz. In some embodiments, each sensor is used as the resonant tank of an oscillator circuit, where the resonant frequency shift indicates the amount of mass loading on the membranes 412. The sensitivity of such a resonator is defined as the ratio of the frequency shift over the frequency: Δf/f=−Δm/2 m, where Δm is the change in mass (i.e., mass of the species that adsorbs or binds to the sensor) over the total mass of the membrane.

In one embodiment, a resonance frequency response of the fundamental mode is supplemented by also measuring a series of higher harmonics of the membrane. The viscoelastic properties of the sensing layer (e.g., polymer) are influenced by absorption/adsorption. These properties are extracted through measuring the frequency dependence of the damping and the amplitude of higher order modes, and these measurements provide chemical information in addition to the resonance frequency. For instance, different mass loadings, polymer swelling and changes in the young modulus are detected through the amplitude and Q-factor. Off-resonance response may also provide information on viscoelasticity through the slope of the mechanical response. In some instances, the membrane in a sensor can be engineered to enhance the response at some harmonics.

In another embodiment, gases or liquids are exposed over a layer of receptor material that adsorbs or binds the molecules of interest. The temperature of exposure will depend on the chemical desorption rate, and may be at room temperature or at lower temperatures depending on the molecule. After a set time, the sensor elements 410 may be heated either by thermal pulse or a linear programmed temperature ramp. During this heating, the molecules are desorbed and the change in resonant frequency and Q-factor shows a particular desorption profile similar to thermal desorption analysis commonly using spectrometric systems or gravimetrically (thermogravimetry). The temperature of desorption is an additional parameter that is sensitive to the chemical nature of the absorbent-absorbate interaction. After one thermal desorption cycle, a second subsequent cycle may be used to provide a reference calibration to be subtracted as a baseline from the first. The second thermal cycle reflects the thermomechanically induced change in resonance frequency.

Referring again to FIG. 2, the use of electrodes 112 provides a convenient method to heat the membrane 110. The small size and structure of the sensors ensures that low energy consumption, low thermal loads and fast (sub millisecond) response times can be achieved. The rapid response times aid resolution in the desorption profile. Temperature readouts of the sensors are also possible through integration of small thermocouples or the resistance of piezoresistive layers. The actual temperature profile during heating can provide information on phase transitions, heating or cooling effects. The sensor structure may be readily optimized to create an array of thermal sensor elements working on the bimetallic effect. Here temperature changes induce both changes in resonant frequency and static bending.

Sensor arrays may be configured, for example, as one-dimensional arrays of sensors or two-dimensional arrays of sensors. An advantage of a two-dimensional array is that an entire wafer may be populated with thousands of sensors. A one-dimensional array provides surface space, which may be used to integrate electronics side-by-side with the sensors. In some embodiments, a two-dimensional sensor array has electronics flip-chip bonded or fabricated under the sensor array. A sensor array with thousands of membranes may be useful in some embodiments for establishing the electrical impedance of the sensor, or for reducing the number of false alarms, if all the membranes in a sensor array are arranged to operate in parallel. If one sensor were to give a false indication, then the other sensors force a correct decision. Having thousands of sensors, many of which are functionalized in the same fashion, can also be used to reduce the false alarm rates and provide a more stable measurement of the presence of one or more analytes.

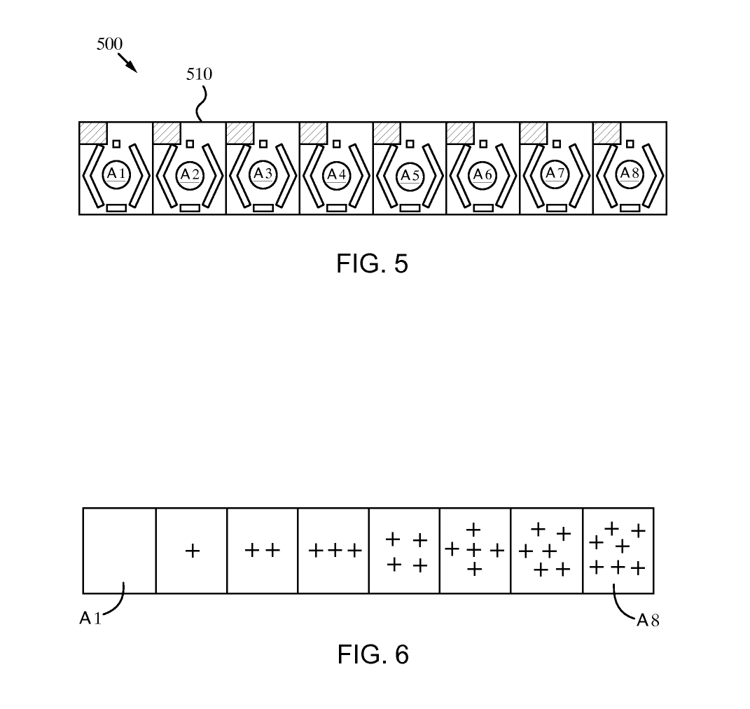

FIG. 5 shows a sensor array 500 having one row of eight resonator sensors 510 functionalized with receptor materials A1-A8. Each of the sensors 510 is functionalized with a different receptor material, and each of the receptor materials A1-A8 has a physical property relevant to its ability to bind or adsorb one or more of the analytes. Suitable physical properties include, but are not limited to, hydrophobicity, hydrophilicity and polarity. The physical property of the receptor materials A1-A8 systematically increases or decreases in degree (e.g., in a pattern or increments) across the array from the receptor material A1 on a first one of the sensors 510 to the receptor material A8 on a last one of the sensors 510.

In some embodiments, the physical property of the receptor materials A1-A8 increases or decreases in degree by substantially equal increments. By way of example, but not limitation, polyvinylidene fluoride is a fluorinated polymer with a monomer unit CH₂CF₂, where the CF₂ unit represents a strong dipole moment. The strong dipole moment attracts gases and organic molecules that carry a dipole moment on their own by virtue of an electrostatic interaction (e.g., alcohols, aldehydes or water). This polymer family may be modified to contain fewer or more fluorine atoms, thereby varying the degree of polarity. By creating a copolymer of the structure (CF₂CH₂)n . . . (CHFC)m, one can vary continuously the number of dipoles in the polymer chain by selecting different values of n and m. In addition to polarity, the degree of hydrophobicity of the receptor materials can be systematically varied by selecting a polymer system containing one or more benzene rings (or lack thereof) for each of the receptor materials A1-A8.

An easy method to increase or decrease the degree of a physical property of the receptor materials A1-A8 (e.g., in substantially equal increments from one sensor in the array to the next) is to blend a functionalized polymer with a non-functional polymer. As an example, polyvinyl alcohol (PVA) has the monomer (C₂H₄O)ₓ. We can vary the degree of polarity (e.g., the number of active PVA monomers on each of the sensors 510) by blending the functionalized polymer with a non-functional polymer. In this embodiment, the receptor material A1 may be a 90/10 mix of the functionalized polymer with non-functional polymer, receptor material A2 may be a 80/20 mix, receptor material A3 may be a 70/30 mix, receptor material A4 may be a 60/40 mix, receptor material A5 may be a 50/50 mix, receptor material A6 may be a 40/60 mix, receptor material A7 may be a 30/70 mix, and receptor material A8 may be a 20/80 mix. There are many other suitable polymer families such as poly (vinyl halides), poly (vinyl alkenes) poly (vinyl ketones), poly (thio ethers), poly (vinyl esters), poly (siloxanes), and ionic liquids which allow the systematic variation of their physical properties via the pathway of copolymers or blended polymers. Possible measurements to determine the systematic increase or decrease in degree of a physical property from one receptor material to the next in the sensor array include, without limitation, the number of fluorine atoms per unit area per sensor or a measurement of the electronegativity according to Pauling or Allen.

Sensors may be functionalized with receptor materials in various ways including the use of ink jet techniques, spotter techniques, microfluidics, self-assembly, shadow masking coupled with the above, or spraying in vacuum through movable mask arrays, to name just a few. In some embodiments, an array of sensors is functionalized with polymers having different properties so that the sensor array can sensitively detect and differentiate chemical compounds, and even complex mixtures. One may select and test an optimum set of polymers as receptor materials to generate a robust signature pattern for an analyte. Polymer receptor materials respond to gas-phase analytes in seconds to tens of minutes. The selection of polymers is preferably optimized to fit the mechanical properties of the resonating elements of the sensors (elasticity, density, thickness, etc.), so that detection time is minimized and sensitivity is maximized.

The sensor surface may be functionalized in a manner that improves the polymer film's stability, control analyte adsorption kinetics, and ease polymer application. For example, functionalization of the membranes may be performed by first coating the exterior surface of the membrane with a metal such as gold that aids adhesion of a receptor material to the surface. The receptor material may be deposited on the surface using various techniques, such as drop ejection, that enable multiple functionalizing liquids to be deposited on the sensor surface, and also reduce or eliminate cross-contamination between adjacent functionalized cells of an array. In some embodiments, neutral polymer gels may be used as carriers for receptor materials. Using this method, a variety of compounds that do not form stable films themselves can be applied through drop or spin coating on a neutral substrate such as silicon dioxide. In order to control the location, applications, volume, and quantity of liquids deposited on the surface, one may use ink jet technology with functionalizing receptor materials instead of inks It is sometimes preferable to use non-thermal deposition technology, if thermal ink jets would harm sensitive fluids. A drop ejector, for example, may be used to deposit the polymer over a sensor. The drop ejector is preferably used to deposit enough drops to cover a sensor. Different ejectors are used for different receptor materials so that adjacent sensors and membranes can be functionalized differently. One deposition technique is to use ultrasound based ejectors where a focused beam evolves a drop from a free surface.

In other embodiments, various other types of receptor materials may be doped or functionalized as required. These materials include, for example, polymers (co-polymers, bio-polymers), sol gels, and porous materials (silicon, zeolite, etc.). DNA, RNA, proteins, cells, bacteria, carbon nanotube arrays, catalysts including metals to enzymes, nanoclusters, organic and inorganic materials including: supramolecules, metal-organic complexes, dendritic materials.

Preferably, each of the receptor materials is a non-specific receptor with respect to the one or more analytes. With this type of non-selective “graded panel” of sensors in the array, one or more target species can react with the sensors with a pattern of masses adsorbed or bound to the sensors, which pattern is specific for each target. The observed pattern can be used to identify a target analyte that has been previously characterized so that its pattern is known. If more than one target analyte is present in a sample (a mixture), then the individual known patterns can be deconvolved to separate the individual analytes. The degree of response can be used to quantify the amount of a particular analyte, e.g. the ppm concentration of a specific gas. If a previously uncharacterized analyte is present, an unknown pattern of masses may be detected. In this case, while a specific identification and quantification of the unknown analyte may not be possible, some general properties of the unknown may be determined from the nature of its pattern. The formation of a spatial pattern of masses on the sensors sorts molecules according to the degree of affinity. So even if the detected analyte is not known, the pattern of masses in the sensor array allows assignment of the analyte to a broader category.

FIG. 6 is a schematic, plan view of a sensor array having receptor materials A1-A8 that systematically increase in degree of polarity across the array The affinity of molecules to a surface may vary depending on the “polar” nature of the surface. A molecule or gas with a strong polarity easily attaches to a surface that has polar groups to establish coulomb interactions. For example, alcohol easily adsorbs on PVA (which contains OH groups). Similarly, a molecule with distributed electrons like benzene has a strong affinity to poly(isobutylene) (PIB). Polymer systems can be tailored with varying degrees of hydrophobicity (using co-polymers for example) to construct an array of sensors with receptor materials having gradual variations in their affinity.

Referring again to FIG. 5, the receptor materials A1-A8 on the eight sensors 510 may systematically vary in polarity. In one example of operation, polyvinyl alcohol (PVA) is used as a receptor material to detect ethanol. Depending on the attach ratio of ethanol to PVA, the mass increase due to analyte on the sensors 510 is determined by the number of ethanol molecules adsorbed on the surface. If we apply a graded panel of sensors in the array where PVA is blended with a non-functional polymer, we can vary the degree of polarity (e.g., the number of active PVA monomers on the sensors 510). As another example, polyisobutylene (PIB) having the monomer CH₂(CH₃)₂ is used in receptor materials to detect a target analyte benzene, C₆H₆.

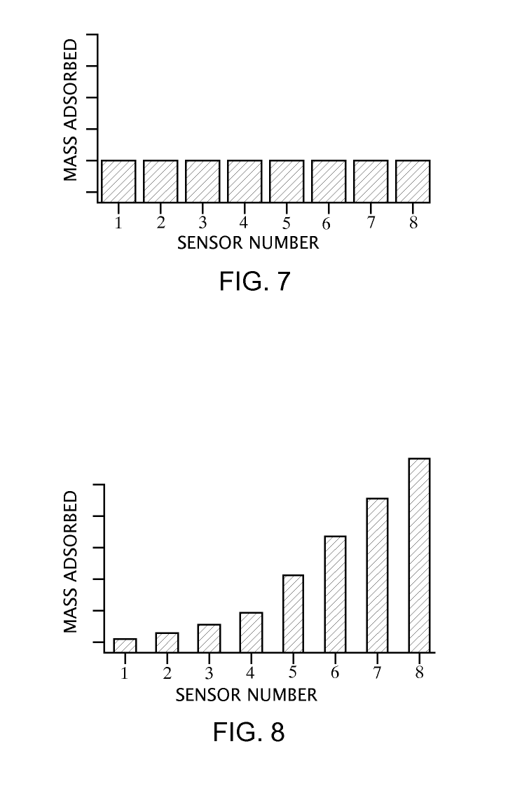

FIGS. 7-8 are graphs showing patterns of masses adsorbed on each functionalized sensor for target analytes. A target molecule like benzene shows little variation from sensors 1-8, as shown in FIG. 7. On the other hand, a polar molecule like ethanol shows a characteristic dependence on the increasing polarity of the receptor materials on sensors 1-8, as shown in FIG. 8. The systematic variation of a physical property of the receptor materials in the sensor array is analogous to information provided by a spectrometer or a chromatograph, and allows additional information to be deduced. One could deduce a charge to mass ratio (e/m ratio), similar to an electrophoretic chromatograph, which is characteristic to a particular molecule or molecule category.

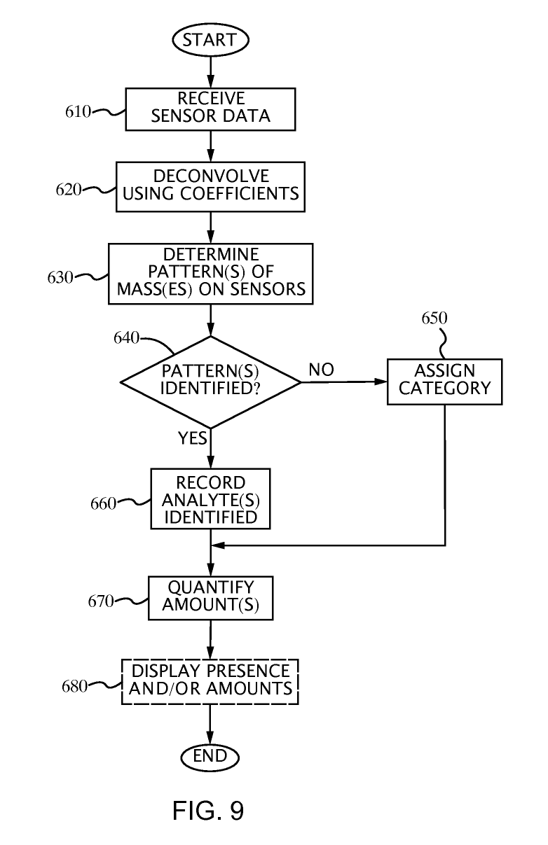

FIG. 9 is a flow chart showing steps of a method employing at least one processor to determine the presence or amount(s) of one or more analytes, according to some embodiments. In step 610, a processor receives data representative of the sensor responses (e.g., changes in resonance frequencies of the functionalized sensors due to mass loading of one or more analytes). In step 620, the processor deconvolves or de-convolutes the data using coefficients. This step can be performed with a set of equations, or more generally by a matrix. In a simple form, let A be the signal amplitude of sensor 1 indicating the sensor response, and X the quantity to of unknown target analyte adsorbed on the sensor 1. We can describe the dependence of amplitude A and unknown quantity X by a linear relationship and a coefficient aₓ so that A=aₓ X. If there is more than one target analyte on sensor 1, such as analyte X and Y, then A=aₓ X+a_{y} Y.

If we now add a second sensor with a different polymer or affinity b_{x and} b_{y}, and assume that the second sensor is exposed to the same quantities X and Y of analytes (since the sensors are adjacent or proximate), then we measure a different value B with second sensor and solve two equations with two variables:

A=aₓX+a_{y}Y (1)

B=bₓX+b_{y}Y (2)

More generally, if we know the matrix of coefficients aᵢⱼ, then we can determine the amounts of multiple analytes Xⱼ if we have measured the amplitudes of I sensors Aᵢ using the vector product (equation 3):

Aᵢ=aᵢⱼ,Xⱼ (3)

If the number of sensors is greater than or equal to the number of targets, the equation can be solved. For example, for a thirty-two sensor chip up to thirty-two different target molecules can be determined. In practice however, one may choose some redundancy to improve accuracy and use thirty-two sensors to target a more limited set of eight analytes. An array of sensors is preferably calibrated to determine the values of the matrix aᵢⱼ, with known analytes of interest Xⱼ. The calibration data is stored either in the sensor array device or in a separate processor where the signals are analyzed.

In step 630, the processor determines respective patterns of masses on the sensors for each analyte (represented in the graphs of FIGS. 7-8 and 11A-11D). In decision step 640, it is determined if one or more of the patterns is identified as representative of a known target analyte. If the pattern is not identified, then the unknown analyte is assigned to a broad category based on its pattern of masses on the sensors, in step 650. If the pattern is identified, then the identified analyte is recorded, in step 660. In step 670, the degree of response is used quantify the amount of one or more analyte(s), e.g., the ppm concentration of a specific gas. In optional step 680, the presence or amounts of the detected analyte(s) are displayed.

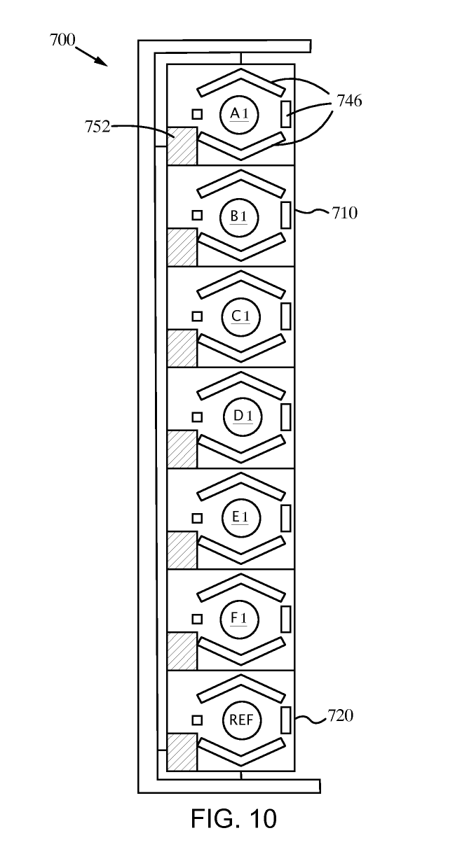

FIG. 10 shows a schematic, plan view of a sensor array 700 according to another embodiment of the invention. Six receptor materials A1-F1 on the sensors 710 systematically increase or decrease in degree of a physical property, such as polarity. In one example, the receptor material A1 on the first sensor comprises polyethylenimine, the receptor material B1 on the second sensor comprises carboxymmethyl cellulose, the receptor material C1 on the third sensor comprises polyethylene glycol, the receptor material D1 on the fourth sensor comprises poly(styrenesulfonate), the receptor material E1 on the fifth sensor comprises polyvinylpyrrolidone, and the receptor material F1 on the sixth sensor comprises poly(methyl methacrylate).

The sensor array 700 may optionally include at least one background or reference sensor 720 to provide a reference signal. The reference sensor 720 includes a non-functionalized membrane instead of a functionalized membrane. The number of sensors in the array that will be used as reference may be easily determined experimentally. Typically, it is expected that 1% to 50% of the sensors in the array will be non-functionalized and used as a reference. The non-functionalized sensors are referred to as a “flat” section of the sensor array while the functionalized sensors are referred to a “graded” section due to the systematic increase or decrease in degree of one or more physical properties of the receptor material. Also, thermistors may be imbedded in the sensor array 700 to provide temperature measurement for temperature compensation. To mechanically isolate each of the sensors 710 or reduce crosstalk between the sensors, vertical trenches 746 may be added between each of the sensors. The trenches 746 may be formed by any known etching process. Each of the sensors 710 may also include wire bond pad areas 752 for electrical connections.

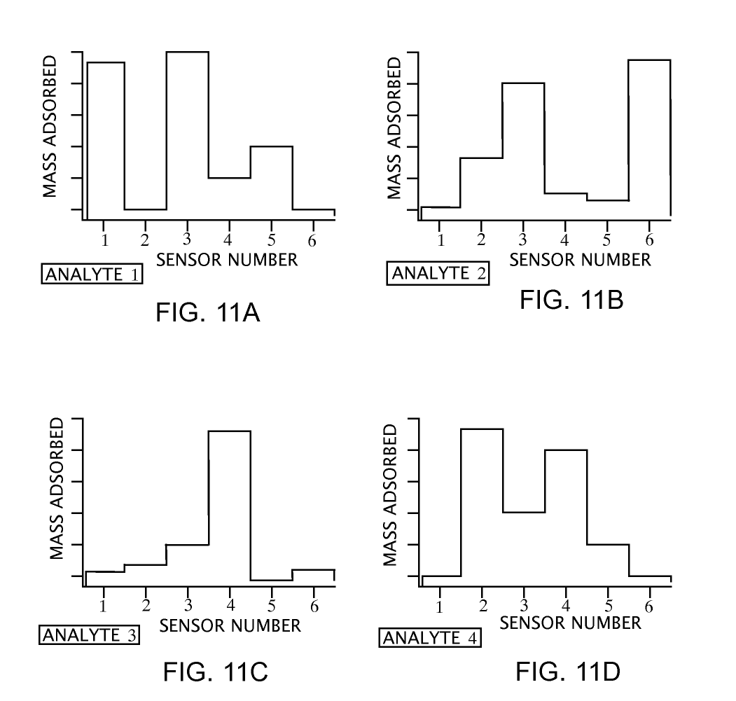

FIGS. 11A-11D are graphs showing patterns of masses adsorbed on each functionalized sensor for four target analytes. In this example, the analytes are volatile organic compounds (VOCs) that adsorb or bind different mass patterns to the six sensors having receptor materials that systematically vary in degree of polarity, as described with reference to FIG. 10. FIG. 11A shows a response pattern of masses of a first analyte, acetaldehyde. FIG. 11B shows a response pattern of a second analyte, benzene. FIG. 11C shows a response pattern of a third analyte, formaldehyde. FIG. 11D shows a response pattern of a fourth analyte, naphthalene. At least one processor may be employed to determine the presence or amount of the analytes, previously described with reference to FIG. 9.

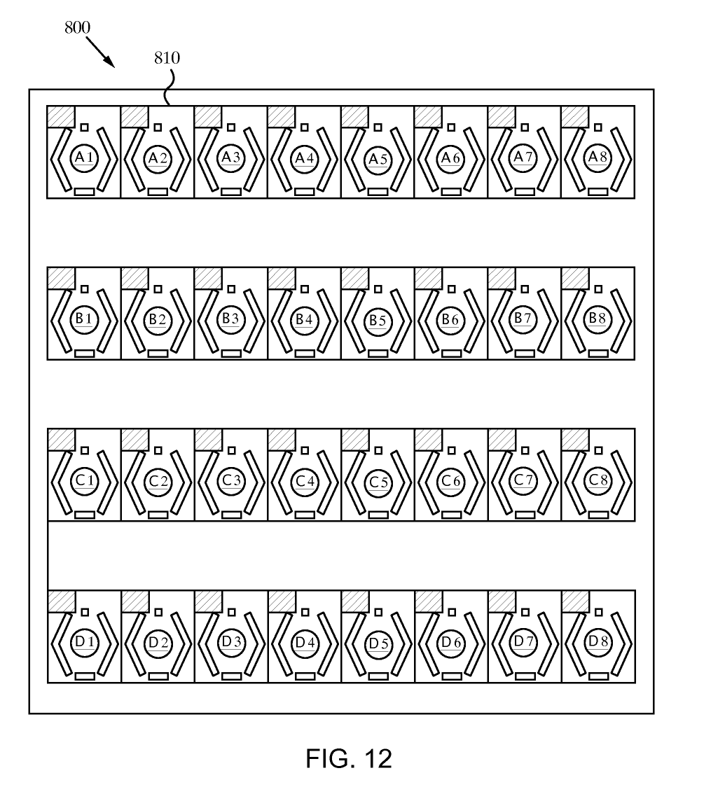

FIG. 12 shows a schematic, plan view of a two-dimensional sensor array 800 having multiple rows and columns according to another embodiment of the invention. The functionalized sensors 810 are arranged in a 4×8 matrix of rows and columns. Each of the receptor materials A1-D8 on the sensors 810 has at least two physical properties (e.g., hydrophobicity, hydrophilicity or polarity) relevant to its ability to bind or adsorb one or more of the analytes. A first one of the physical properties of the receptor materials A1-D8 on the sensors 810 systematically increases or decreases in degree from the first row to the last row, and a second one of the physical properties of the receptor materials A1-D8 on the sensors 810 systematically increases or decreases in degree from the first column to the last column. An example for this design is using different blends of PVA as a receptor material to bind or adsorb polar molecules and different blends of PIB as a receptor material to adsorb or bind benzene, toluene, etc. In one embodiment of the sensor array 800, the polarity varies in degree from receptor material A1 to D1, whereas the hydrophobicity of the receptor materials varies in degree from receptor materials A1 to A8, with receptor material A8 being the most hydrophobic and receptor material A1 being the most hydrophilic.

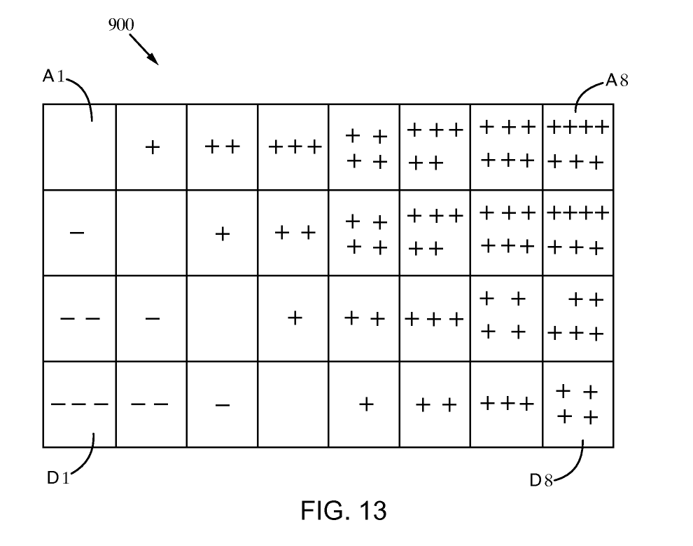

FIG. 13 is a schematic, plan view of a sensor array 900 having receptor materials A1-D8 with positive and negative charges, according to some embodiments of the invention. Suitable receptor materials include blends of polysiloxane which has amine groups that attract negatively charged groups, and blends of poly vinylidene fluoride which carries fluorine atoms that attract positively charged groups. At least one processor may be employed with sensor array 900 to determine the presence or amount of the analytes adsorbed or bound to the receptor materials A1-D8, previously described with reference to FIG. 9.

Sensor arrays may be made with any of various known CMUT fabrication techniques including: SOI bonding, sacrificial layer, surface or bulk micromachining, and silicon on insulator bonding. The metal on the membrane is chosen to ensure the adhesion of the functionalizing receptor material. The sensor is preferably designed for maximum sensitivity while taking into consideration its mechanical loading and electrical interfacing into the integrated (or non-integrated) electronic circuitry. Sensors may be integrated with electronics in any of various known configurations including: flip chip bonding, elements constructed on top of electronics, or vice versa. The sensors may be fabricated with through wafer vias or trench isolated by etching through the back side using various well-known techniques for cMUT fabrication. Techniques suitable for fabricating such sensors are known in the art and are described, for example, in B. T. Khuri-Yakub and L. Levin, U.S. Pat. No. 5,828,394, which is incorporated herein by reference.

Sensor arrays may be used in liquid, gas, or vacuum. Operational temperatures range from cryogenic to high temperatures, depending on the limits of the receptor materials. For operation in immersion, the cells in the cMUT or pMUT or mMUT, or whatever variation, could be made with cells that are distant from nearest neighbors, by design, to provide a narrow band operation. In one embodiment, sensor arrays may be connected down stream from a separation system (such as used in chromatography or gel electrophoresis) to detect specific patterns in complex mixtures of liquids. In this embodiment, relatively simple baffle structures created on chip may provide a further analytical signal input for the device to learn chemicals through processing of the relative time delays caused by different diffusion properties of chemicals sensed between individual sensor elements on one chip or between a plurality of chips. The principles of such devices may use the human nose as a model, which has a duplicity of sensors where the time delays are used in the smelling process.

In operation, a sensor array can be mounted on a wall, ceiling or other portion of a fixed structure, incorporated into a hand-held device, or mounted on a moving vehicle, to name just a few methods of exposing the sensor array to a sample. Depending on the specific application, it may be used with or without active circulation of analyte-containing gas or liquid over the sensors to increase exposure of the sensor to analytes (e.g., chemicals) in the environment. A general guideline for high sensitivity in detection of small quantities of materials is to position the sensor as close to the sampling inlet as possible. The small dimensions of the sensor arrays readily facilitate the integration of the sensor at even millimeter distances from the sampling inlet. Ring arrays may be made with sensor elements that are 30 microns in diameter and where nine sensor elements are connected together to form a sensor, thus making a sensor that 100 microns by 100 microns in size. Resonant devices may be made with sub-100 micron dimensions.

In liquids, the sensor arrays can be incorporated into submarines, ships, divers' handheld devices or parts. Likewise, drinking water, toilets and anywhere liquids are used can be readily monitored or tracked. In another mode of operation, sensor arrays may be used as part of microfluidic devices enabling the detection of small (μL) micro liter volumes. The use of artificial Q enhancements can be applied to increase the sensitivity of the apparatus. Sensor arrays may be integrated with other devices and systems such as, for example, gas handling systems or self-calibrating systems.

It will be clear to one skilled in the art that the above embodiments may be altered in many ways without departing from the scope of the invention. Many different permutations or arrangements may be used to realize the device and method of the invention. For example, sensor arrays containing multiple sensors may have membranes with different resonant frequencies. A membrane operating at low frequency yields a sensor more sensitive to stress on the membrane, whereas a membrane operating at high frequency gives a sensor that is more sensitive to mass loading. Combining various operating frequencies in one sensor thus provides a sensor with a greater versatility.

In some embodiments, the amount of receptor material used to functionalize each sensor may be varied. For example, the number of droplets of receptor material placed on each sensor can be varied from one sensor to the next, thereby varying the thickness of the deposited receptor material. This variation in the amount or thickness of receptor material on each sensor establishes one more pathway to provide redundancy and enhance the accuracy of the sensor array (fewer false positives).

In some embodiments, electronics are integrated with sensor arrays, where multiple sensors are attached in parallel, and sensors are operated at different frequencies so that one output line may be used. For this purpose, different sensors may be built and operated at different frequencies. For example, a row of sensors can be made to resonate from 45 MHz to 55 MHz in 0.1 MHz intervals. Principles of dense wavelength division multiplexing (DWDM) may be used in such devices. A sensor for a Dog Nose type sensor may be made of one of multiple capacitor membranes that are all attached in parallel by virtue of having a metal electrode that covers all the sensors partially or fully. By altering the diameter of resonating elements, it is possible to change the frequency of operation. Having sensors operating at multiple frequencies can have advantages in electronic integration in transmitting information at different frequencies on the same channel, and in separating the influence of stress and mass loading on the shift in resonant frequency of a resonator.

In one embodiment, large arrays of sensors may be used to develop a physics/chemistry based model to extract unique inversion for single element identification using multiple functional agents. In particular, collecting data about the sensitivity to certain species by different functionalizing chemistries allows the development of a model to relate the outputs of the multiple sensors to the different species with higher accuracy.

In some embodiments, arrays of sensors are functionalized by a wide range of receptor materials. For example, for polymer receptor materials, some 500 polymers with a redundancy factor of 10 may be used. The specific responses including orthogonality of response, operation mode (temperature, integration times, etc.), lifetime and sensitivity of environment or other disrupting influences may be tested using the target molecules and interfering agents. Based on a self-optimization the system may then select the most sensitive polymer basis set (say 10 polymers) and optimum mode of operation. In this way, different customers, corporate (food, perfume), medical (breath, urine, blood analysis), security or military can obtain rapidly prototyped solutions. Incorporation of this data in a database for future development of prototypes and known response functions can be used.

In some embodiments, sensor arrays have a vast number of independently addressed sensors in the array to provide a massive redundancy. For instance, in an array of 5000 sensors, one can have a redundancy factor of 100 using 50 receptor and reference materials. This ensures that false alarms or defective elements in the arrays which might miss the detection of analytes are not an issue in device operation. The self calibration and learning feature of such arrays is also a mode which takes full advantage of redundancy. It permits defective elements and the control quarantine and analytical potential of the device to be optimized on the fly. Furthermore, it permits new threatening chemicals that may be identified to be quickly introduced into the detection capabilities of machines installed at different operational locations.

Accordingly, the scope of the invention should be determined by the following claims and their legal equivalents.

What is claimed is:

1. A device for detecting one or more analytes, the device comprising:
a) a sensor array including at least three resonator sensors having respective receptor materials disposed thereon, wherein the receptor materials have a physical property relevant to their ability to bind or adsorb one or more of the analytes, and the physical property of the receptor materials systematically increases or decreases in degree from the receptor material on a first one of the sensors, to the receptor material on a second one of the sensors, and to the receptor material on a third one of the at least three sensors; and
b) detection means for detecting sensor responses when masses of one or more of the analytes are adsorbed or bound to the receptor materials on the sensors.

2. The device of claim 1, wherein the physical property of the receptor materials comprises hydrophobicity or hydrophilicity.

3. The device of claim 1, wherein the physical property of the receptor materials comprises polarity.

4. The device of claim 1, wherein the physical property of the receptor materials increases or decreases in degree by substantially equal increments.

5. The device of claim 1, wherein each of the receptor materials is a non-specific receptor with respect to the one or more analytes.

6. The device of claim 1, wherein the sensor responses comprise changes in resonance frequencies, and the detection means comprises at least one detector for detecting resonance frequencies of the sensors.

7. The device of claim 1, further comprising at least one processor in communication with the detection means for receiving data representative of the sensor responses, wherein the at least one processor is programmed to determine the presence or amount of one or more of the analytes according to the data.

8. The device of claim 7, wherein the at least one processor is programmed to determine the presence or amount of one or more of the analytes by determining the respective masses of the analytes on the sensors according to the data and by determining the presence or amount of one or more of the analytes according to at least one pattern of the masses on the sensors.

9. The device of claim 1, further comprising at least one processor in communication with the detection means for receiving data representative of the sensor responses, wherein the at least one processor is programmed to determine respective amounts of the analytes on the sensors by de-convolution of the data.

10. The device of claim 9, wherein the processor is programmed to determine the amounts using coefficients relating the sensor responses to the respective masses of the analytes on the sensors.

11. The device of claim 1, wherein each of the resonator sensors comprises a capacitive micromachined ultrasound transducer (cMUT).

12. The device of claim 1, wherein the sensor array includes at least six resonator sensors having respective receptor materials disposed thereon, wherein the physical property of the receptor materials on the at least six sensors systematically increases or decreases in degree from the receptor material on the first one of the sensors to the receptor material on a sixth one of the sensors.

13. The device of claim 1, wherein the sensor array further includes at least one reference sensor lacking a receptor material.

14. A device for detecting one or more analytes, the device comprising:
a) a sensor array including a plurality of resonator sensors having respective receptor materials disposed thereon, wherein the sensors are arranged in rows and columns, the receptor materials have a first physical property relevant to their ability to bind or adsorb one or more of the analytes, the first physical property of the receptor materials on the sensors in at least one of the rows systematically increases or decreases in degree from the receptor material on a first one of the sensors in the row to the receptor material on a last one of the sensors in the row, and the first physical property or a second physical property of the receptor materials on the sensors in at least one of the columns systematically increases or decreases in degree from the receptor material on a first one of the sensors in the column to the receptor material on a last one of the sensors in the column; and
b) detection means for detecting sensor responses when masses of one or more of the analytes are adsorbed or bound to the receptor materials on the sensors.

15. The device of claim 14, wherein the first physical property of the receptor materials comprises hydrophobicity or hydrophilicity, and the second physical property of the receptor materials comprises polarity.

16. The device of claim 14, wherein the first physical property of the receptor materials systematically increases or decreases in degree by substantially equal increments from the receptor material on one sensor to the next in the row, and the second physical property of the receptor materials systematically increases or decreases in degree by substantially equal increments from the receptor material on one sensor to the next in the column.

17. The device of claim 14, wherein each of the receptor materials is a non-specific receptor with respect to the one or more analytes.

18. The device of claim 14, wherein the sensor responses comprise changes in resonance frequencies, and the detection means comprises at least one detector for detecting resonance frequencies of the sensors.

19. The device of claim 14, further comprising at least one processor in communication with the detection means for receiving data representative of the sensor responses, wherein the at least one processor is programmed to determine the presence or amount of one or more of the analytes according to the data.

20. The device of claim 19, wherein the at least one processor is programmed to determine the presence or amount of one or more of the analytes by determining respective masses of the analytes on the sensors according to the data and by determining the presence or amount of one or more of the analytes according to at least one pattern of the masses on the sensors.

21. The device of claim 14, further comprising at least one processor in communication with the detection means for receiving data representative of the sensor responses, wherein the at least one processor is programmed to determine respective amounts of the analytes on the sensors by de-convolution of the data.

22. The device of claim 21, wherein the processor is programmed to determine the amounts using a matrix of coefficients relating the sensor responses to respective masses of the analytes on the sensors.

23. The device of claim 14, wherein each of the resonator sensors comprises a capacitive micromachined ultrasound transducer (cMUT).

24. The device of claim 14, wherein the sensor array further includes at least one reference sensor lacking a receptor material.

25. A method comprising:
a) exposing a sensor array to a sample that potentially contains one or more analytes, wherein the sensor array includes at least three resonator sensors having respective receptor materials disposed thereon, wherein the receptor materials have a physical property relevant to their ability to bind or adsorb one or more of the analytes, and the physical property of the receptor materials systematically increases or decreases in degree from the receptor material on a first one of the sensors, to the receptor material on a second one of the sensors, and to the receptor material on a third one of the at least three sensors;
b) detecting sensor responses to the sample; and
c) determining from the sensor responses if the sample contains one or more of the analytes.

26. The method of claim 25, wherein the physical property of the receptor materials comprises hydrophobicity or hydrophilicity.

27. The method of claim 25, wherein the physical property of the receptor materials comprises polarity.

28. The method of claim 25, wherein the physical property of the receptor materials increases or decreases in degree by substantially equal increments.

29. The method of claim 25, wherein each of the receptor materials is a non-specific receptor with respect to the one or more analytes.

30. The method of claim 25, wherein the sensor responses comprise changes in resonance frequencies, and the detection means comprises at least one detector for detecting resonance frequencies of the sensors.

31. The method of claim 25, further comprising the steps of employing at least one processor to receive data representative of the sensor responses and to determine the presence or amount of one or more of the analytes according to the data.

32. The method of claim 31, wherein the at least one processor is programmed to determine the presence or amount of one or more of the analytes by determining respective masses of the analytes on the sensors according to the data and by determining the presence or amount of one or more of the analytes according to at least one pattern of the masses on the sensors.

33. The method of claim 25, further comprising the steps of employing at least one processor to receive data representative of the sensor responses and to determine respective amounts of the analytes on the sensors by de-convolution of the data.

34. The method of claim 33, wherein the at least one processor determines the amounts using coefficients relating the sensor responses to respective masses of the analytes on the sensors.

35. The method of claim 25, wherein each of the resonator sensors comprises a capacitive micromachined ultrasound transducer (cMUT).

36. The method of claim 25, wherein the sensor array includes at least six resonator sensors having respective receptor materials disposed thereon, wherein the physical property of the receptor materials on the at least six sensors systematically increases or decreases in degree from the receptor material on the first one of the sensors to the receptor material on a sixth one of the sensors.

37. A method comprising:
a) exposing a sensor array to a sample that potentially contains one or more analytes, wherein the sensor array includes a plurality of resonator sensors having respective receptor materials disposed thereon, wherein the sensors are arranged in rows and columns, the receptor materials have a first physical property relevant to their ability to bind or adsorb one or more of the analytes, the first physical property of the receptor materials on the sensors in at least one of the rows systematically increases or decreases in degree from the receptor material on a first one of the sensors in the row to the receptor material on a last one of the sensors in the row, and the first physical property or a second physical property of the receptor materials on the sensors in at least one of the columns systematically increases or decreases in degree from the receptor material on a first one of the sensors in the column to the receptor material on a last one of the sensors in the column;
b) detecting sensor responses to the sample; and
c) determining from the sensor responses if the sample contains one or more of the analytes.

38. The method of claim 37, wherein the first physical property of the receptor materials comprises hydrophobicity or hydrophilicity, and the second physical property of the receptor materials comprises polarity.

39. The method of claim 37, wherein the first physical property of the receptor materials systematically increases or decreases in degree by substantially equal increments from the receptor material on one sensor to the next in the row, and the second physical property of the receptor materials systematically increases or decreases in degree by substantially equal increments from the receptor material on one sensor to the next in the column.

40. The method of claim 37, wherein each of the receptor materials is a non-specific receptor with respect to the one or more analytes.

41. The method of claim 37, wherein the sensor responses comprise changes in resonance frequencies, and the detection means comprises at least one detector for detecting resonance frequencies of the sensors.

42. The method of claim 37, further comprising the steps of employing at least one processor to receive data representative of the sensor responses and to determine the presence or amount of one or more of the analytes in dependence upon the data.

43. The method of claim 42, wherein the at least one processor is programmed to determine the presence or amount of one or more of the analytes by determining respective masses of the analytes on the sensors according to the data and by determining the presence or amount of one or more of the analytes according to at least one pattern of the masses on the sensors.

44. The method of claim 37, further comprising the steps of employing at least one processor to receive data representative of the sensor responses and to determine respective amounts of the analytes on the sensors by de-convolution of the data.

45. The method of claim 44, wherein the at least one processor determines the amounts using a matrix of coefficients relating the sensor responses to respective masses of the analytes on the sensors.

46. The method of claim 37, wherein each of the resonator sensors comprises a capacitive micromachined ultrasound transducer (cMUT).
